(19) United States
(12) Patent Application Publication
(10) Pub. No.: US 2026/0354667 A1
(43) Pub. Date: Nov. 12, 2026

(54) COUPLING MECHANISMS FOR MEDICAL DEVICES AND RELATED METHODS

(71) Applicant: Boston Scientific Scimed, Inc., Maple Grove, MN (US)

(72) Inventors: Daniel E. WEDDLE, Freedom, IN (US); Vincent Michael ROSSODIVITO, Bowling Green, IN (US); Thomas M. RAGONESE, Bloomington, IN (US); Jessica DeVILLERS, Bloomington, IN (US); Michael RATZ, Avon, IN (US)

(73) Assignee: Boston Scientific Scimed, Inc., Maple Grove, MN (US)

(21) Appl. No.: 19/668,231

(22) Filed: May 5, 2026

Related U.S. Application Data
(60) Provisional application No. 63/801,220, filed on May 7, 2025.

(51) Int. Cl. A61B 1/00

(52) U.S. Cl. CPC A61B 1/00128; A61B 1/00066; A61B 1/00119

(57) ABSTRACT

Medical devices and related methods are described. The medical device may include a handle having a distal portion, a shaft having a proximal end configured to engage with the distal portion of the handle, and a coupling mechanism including a lock configured to receive the distal portion of the handle to secure the shaft and the lock to the handle.

### CROSS-REFERENCE TO RELATED APPLICATIONS

This application claims the benefit of priority from U.S. Provisional Application No. 63/801,220, filed on May 7, 2025, which is incorporated by reference herein in its entirety.

### TECHNICAL FIELD

The present disclosure relates to medical devices with coupling mechanisms to securely attach a handle to a shaft. The coupling mechanism may include a lock.

### BACKGROUND

Medical devices having an elongated shaft or insertion portion permanently fixed to a handle are used in healthcare settings for diagnostic and therapeutic procedures. Traditional methods of attaching an insertion portion to a handle may include various manufacturing methods resulting in the permanent fixation between the two components. The manufacturing methods such as overmolding, insert molding, or adhesive bonding often suffer from inconsistency due to variations in materials, tolerances, environmental factors, and process parameters. These inconsistencies can lead to various challenges including manufacturing inefficiencies, increased rejection rates, and potential performance issues during clinical use. Furthermore, users are unable to interchange insertion portions with a handle in these types of medical devices.

### SUMMARY

The present disclosure relates to, among other things, medical devices having coupling mechanisms to attach handles to shafts and related methods. Each aspect disclosed herein may include one or more features described in connection with any other disclosed aspect.

According to aspects described herein, the medical device includes a handle having a distal portion; a shaft having a proximal end configured to engage with the distal portion of the handle; and a coupling mechanism including a lock configured to receive the distal portion of the handle to secure the shaft and the lock to the handle.

The medical devices described herein may include any or all of the following features in any combination.

The distal portion of the handle may include a tapered distal end and/or the proximal end of the shaft may include a flared portion. The flared portion may be configured to abut the tapered distal end of the handle. The flared portion may include, for example, one or more cutouts extending longitudinally along at least a portion of the flared portion. The flared portion may include one or more cutouts configured to receive a protrusion of the distal portion of the handle.

In some examples, the lock of the coupling mechanism includes a central opening configured to receive both the shaft and the handle. The handle may be attached to the shaft by the coupling mechanism, and the proximal end of the shaft may be disposed between an internal surface of the lock and the distal portion of the handle. The lock may include one or more indentations configured to engage with a protrusion of the distal portion of the handle to secure the lock to the handle. According to some aspects, the lock may include one or more slits extending longitudinally along at least a portion of the lock. Further, in some examples, the distal portion of the handle includes threads complementary to threads of the lock. The lock may include a proximal-facing recess configured to receive the distal portion of the handle. For example, the distal portion of the handle may include a distal-facing recess.

Optionally, the coupling mechanism further includes one or more of an O-ring, a spacer, or a washer. In some examples, the spacer is between the washer and the O-ring. The coupling mechanism may further include an O-ring and a crimp ring, e.g., the crimp ring being distal of the O-ring.

The handle may include a first lumen extending longitudinally therethrough and/or the shaft may include a second lumen extending longitudinally therethrough. For example, upon securing the shaft to the handle, the first lumen may be in fluid communication with the second lumen.

The present disclosure also includes a medical device comprising a handle having a distal portion with a tapered distal end, a shaft having a proximal end with a flared portion that receives the tapered distal end of the handle, and a coupling mechanism including a lock that secures the shaft to the handle. The flared portion of the shaft may be between an internal surface of the lock and the tapered distal end of the handle.

The flared portion may include one or more cutouts extending longitudinally along at least a portion of the flared portion. In some examples, the shaft may be detachable from the handle, e.g., by manipulating the coupling mechanism to detach the lock from the handle or the shaft.

According to some aspects described herein, the medical device includes a handle having a distal portion with a tapered distal end, a shaft having a proximal end with a flared portion that receives the tapered distal end of the handle, and a coupling mechanism including a lock, an O-ring, a spacer, and a washer. The coupling mechanism secures the shaft to the handle. Optionally, the spacer may be between the washer and the O-ring.

### BRIEF DESCRIPTION OF FIGURES

The accompanying drawings, which are incorporated in and constitute a part of this application, illustrate aspects of this disclosure and together with the description, serve to explain the principles of this disclosure.

FIG. 1A illustrates an exploded view of an exemplary medical device, according to aspects of this disclosure.

FIG. 1B illustrates an assembled cross-sectional view of the exemplary medical device of FIG. 1A, according to aspects of this disclosure.

FIG. 1C illustrates a perspective view of a portion of the exemplary medical device of FIGS. 1A and 1B, according to aspects of this disclosure.

FIG. 2A illustrates an exploded view of another exemplary medical device, according to aspects of this disclosure.

FIG. 2B illustrates an assembled cross-sectional view of the exemplary medical device of FIG. 2A, according to aspects of this disclosure.

FIGS. 3A and 3B illustrate cross-sectional views of another exemplary medical device, according to aspects of this disclosure.

FIGS. 4A and 4B depict another exemplary medical device, according to aspects of this disclosure.

### DETAILED DESCRIPTION

Both the foregoing general description and the following detailed description are exemplary and explanatory only and are not restrictive of the features, as claimed. As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range. The terms “proximal” and “distal” are used herein to refer to the relative positions of the components of exemplary medical devices (see proximal “P” and distal “D” directional arrows throughout various figures). As used herein, “proximal” refers to a position relatively closer to the exterior of the body or closer to an operator using the medical device. In contrast, “distal” refers to a position relatively further away from the operator using the medical device, or closer to the interior of the body.

Aspects of the present disclosure are described herein with reference to exemplary medical devices and coupling mechanisms thereof that may be useful for attaching and/or detaching a shaft to a handle. The medical devices described herein may include a shaft (alternatively referred to as an insertion portion) configured to attach to and/or detach from a handle of the medical device. The shaft may be secured to the handle via a coupling mechanism, which may include a lock. In some aspects, the ability to attach/detach the shaft from the handle may allow for increased flexibility in component selection and utilization. For example, medical professionals may have access to a range of shaft lengths or types that can be interchanged with a single handle. This modular approach may allow for the use of different shaft configurations for various procedures or patient anatomies while maintaining familiarity with a consistent handle design. Aspects of this disclosure may allow for the handle to be used and sterilized multiple times, e.g., after detaching the shaft following a medical procedure. For example, the shaft may be used once for a single patient while the handle, not contacting the patient, may be used with different shafts.

Additionally or alternatively, the medical devices described herein may allow for more efficient production processes and potentially reduce overall manufacturing costs. For example, the interaction between components described herein may also contribute to manufacturing efficiencies. In some aspects, the coupling mechanism(s) may allow for increased tolerances in component manufacturing, potentially simplifying production processes and/or reducing material waste.

References to a particular type of procedure, such as a urology procedure; a medical device or medical instrument, such as a catheter or scope; and/or an organ, such as a kidney, are provided for convenience and not intended to limit this disclosure. Accordingly, analogous medical devices or systems may utilize one or more aspects of the exemplary medical devices described herein.

Reference is now made in detail to examples to help illustrate aspects of the present disclosure through the accompanying drawings. Wherever possible, the same or similar reference numbers will be used throughout the drawings to refer to the same or like parts. Any of the devices disclosed herein may include any of the following features, additionally or alternatively, in any combination. Accordingly, between different embodiments, like numbers will be used to refer to like features, with “100” added to each numeral.

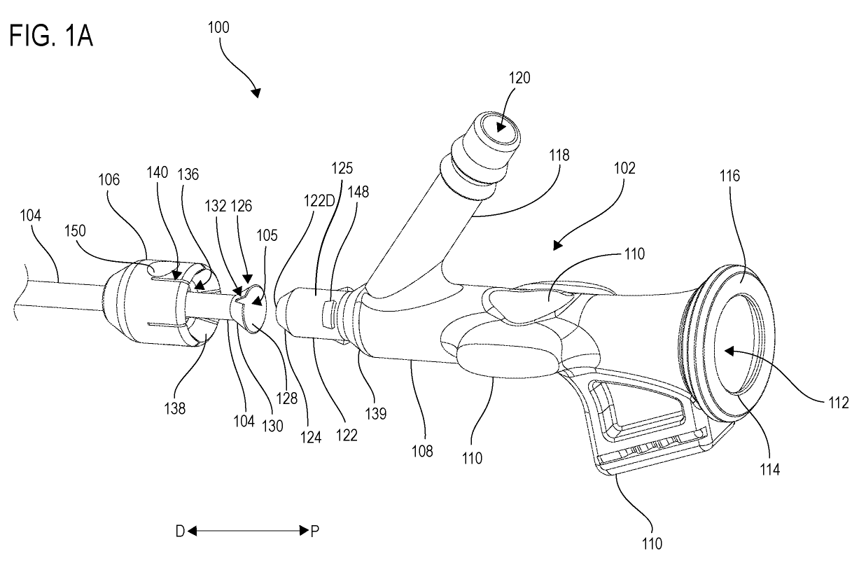

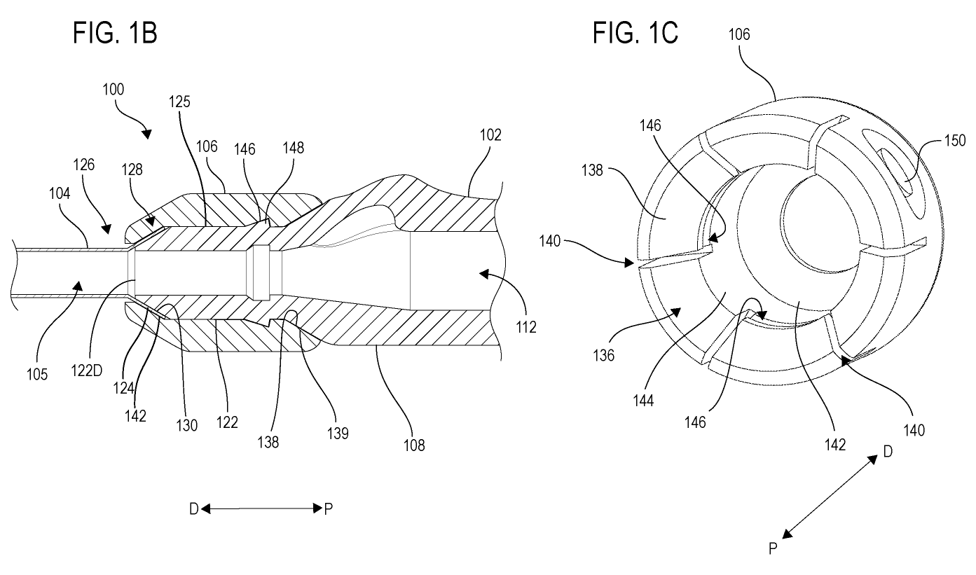

FIGS. 1A-1C illustrate various views of at least a portion of an exemplary medical device 100 according to aspects of this disclosure. For example, FIG. 1A illustrates an exploded view of medical device 100, which includes a handle 102 and a shaft 104. Described in further detail below, shaft 104 may be configured to attach to and/or detach from handle 102 via a coupling mechanism. For example, the coupling mechanism may include a lock 106 configured to secure shaft 104 to handle 102. FIG. 1B illustrates an assembled cross-sectional view of medical device 100, revealing the internal structure and how shaft 104 and lock 106 interfaces with handle 102 and with each other. FIG. 1C depicts a perspective view of lock 106.

Referring to FIG. 1A, handle 102 may include a gripping portion 108. Gripping portion 108 may be configured to allow a user to comfortably hold and manipulate handle 102 to control medical device 100. In some aspects, gripping portion 108 may include one or more surface features 110. These surface features 110 may enhance the user's grip on the handle 102 (e.g., promoting friction between the user’s hand and gripping portion 108) and may take various forms such as textured surfaces, contoured shapes, indentions, protrusions, and/or other ergonomic designs.

Handle 102 may include one or more lumens 112 extending from a proximal end of handle 102 to a distal end of handle 102. For example, handle lumen(s) 112 may extend through at least a portion of handle 102 and may be configured to receive fluids (e.g., a liquid or gas) and/or auxiliary medical instruments (e.g., biopsy forceps, guidewires, snares, baskets, etc.). Described in further detail below, lumen 112 may be aligned and in fluid communication with one or more lumens 105 of shaft 104. In some aspects, a proximal opening 114 of lumen 112 may be disposed on a proximal-most face 116 of handle 102. Proximal opening 114 may serve as an entry point for inserting the auxiliary medical instrument(s) into handle 102 and/or for applying or removing fluid(s) from lumen 112. In other aspects, lumen 112 may be closed (e.g., proximal opening 114 may be omitted). In some aspects, lumen 112 may be referred to herein as a first lumen or a handle lumen.

Handle 102 may further include a port 118. Port 118 may include an opening 120 that is in fluid communication with lumen 112 of handle 102. In some aspects, a longitudinal axis of port 118 may form an angle, e.g., acute angle, relative to a longitudinal axis of lumen 112 of handle 102. Port 118 may be configured to receive fluids (e.g., liquids or gases) and/or an auxiliary medical instrument (e.g., biopsy forceps, guidewire, etc.). In some aspects, a fluid source may be coupled directly or indirectly to port 118.

Handle 102 may include a distal portion 122 extending from a distal end of gripping portion 108. For example, distal portion 122 may be distal of gripping portion 108 and port 118. A distal opening of lumen 112 may be disposed on a distal-most surface 122D of distal portion 122. In some aspects, distal portion 122 may have a generally cylindrical shape having a smaller cross-sectional dimension (e.g., smaller diameter) as compared to portions of handle 102 that are proximal of distal portion 122 (e.g., gripping portion 108). Distal portion 122 may include a distal end 124, a proximal end 139, and a central portion 125 therebetween. Central portion 125 may have a uniform cross-sectional dimension (e.g., constant diameter), thereby forming a substantially straight or uniform portion of distal portion 122. Each of distal end 124 and proximal end 139 may taper distally. For example, a cross-sectional dimension (e.g., diameter) of each of distal end 124 and proximal end 139 may gradually decrease proximally to distally. In these aspects, each of distal end 124 and proximal end 139 optionally may have a conical or frustoconical shape.

Distal end 124 may be configured to facilitate engagement with a proximal end 126 of shaft 104. For example, distal end 124 may be complimentarily shaped relative to proximal end 126. Proximal end 126 of shaft 104 may include a flared portion 128 having an internal surface 130. Proximal end 126 of shaft 104 may define a proximal end of one or more lumens 105 of shaft 104. Lumen(s) 105 of shaft 104 may extend from proximal end 126 of shaft 104 to a distal end of shaft 104. A distal opening of lumen(s) 105 may be disposed on a distal-most end of shaft 104 (and/or at any location along shaft 104 that is proximal of distal-most end of shaft 104).

Proximal end 126 of shaft 104 (e.g., flared portion 128) may be configured to receive at least a portion of distal end 124 of handle 102 such that internal surface 130 abuts or otherwise engages with distal end 124 of handle 102. For example, internal surface 130 may abut or otherwise be pressed against distal end 124. This interaction may create a close fit between proximal end 126 of shaft 104 and distal end 124 of handle 102. In some aspects, the interaction between proximal end 126 of shaft 104 and distal end 124 of handle 102 may result in one or more continuous lumens between handle 102 and shaft 104. For example, upon assembly, lumen(s) 112 of handle 102 and lumen(s) 105 of shaft 104 may be in fluid communication with each other. This alignment of lumens may allow for uninterrupted passage of fluids, gases, and/or medical instruments from handle 102 through shaft 104.

Flared portion 128 may include one or more cutouts 132 (e.g., slit(s) or notch(es)) extending longitudinally along at least a portion of flared portion 128. Cutout(s) 132 together with the material(s) of which flared portion 128 is formed may allow for some flexibility or expansion of flared portion 128 during assembly. For example, cutout(s) 132 may permit flared portion 128 of shaft 104 to expand to receive distal end 124 of handle 102. Such a configuration may assist in ensuring a close fit therebetween. Exemplary materials suitable for flared portion 128 and other components of medical device 100 include, but are not limited to, biocompatible polymers. In other aspects, cutout(s) 132 may be omitted. In such aspects, the material(s) of flared portion 128 of shaft 104 may provide flexibility. For example, flared portion 128 may comprise a flexible or semi-flexible material (e.g., polymer) so as to flex or expand to receive distal end 124 of handle 102.

Additionally or alternatively, cutout(s) 132 may be configured to receive an alignment feature of distal end 124 of handle 102. The alignment feature of distal end 124 may be configured to assist with the alignment of the shaft 104 relative to handle 102 and/or maintain their relative positions during use. For example, cutout(s) 132 may be configured to receive complementary protrusion(s) of distal end 124. In such aspects, cutout(s) 132 may be configured to inhibit or prevent shaft 104 from twisting or torquing relative to handle 102, thereby maintaining a position of shaft 104 relative to handle 102.

Referring primarily to FIGS. 1A and 1C, the coupling mechanism for attaching handle 102 to shaft 104 includes a lock 106. Lock 106 may include a central opening 136 configured to receive shaft 104 and at least a portion of distal portion 122 of handle 102. For example, lock 106 may be configured to secure shaft 104 to handle 102, thereby maintaining the connection between these components during use of medical device 100. In some aspects, lock 106 may be designed to position proximal end 126 of shaft 104 between an internal distal surface 142 of lock 106 and distal end 124 of handle 102, which may facilitate engagement between shaft 104 and handle 102.

Lock 106 may include one or more engagement features 146 configured to interact with a complementary engagement feature 148 of distal portion 122. For example, engagement feature 146 of lock 106 may take the form of a protrusion or an indentation, and complementary engagement feature 148 of distal portion 122 may take the form of a complementary indentation or protrusion. Through this interaction, lock 106 may be secured to handle 102 via engagement features 146, 148. In these aspects, the coupling mechanism may secure shaft 104 to handle 102 via lock 106.

Lock 106 may further include a proximal internal surface 138. In some aspects, proximal internal surface 138 of lock 106 may be configured to engage with, e.g., abut, proximal end 139 of handle 102. The interaction between proximal internal surface 138 of lock 106 and proximal end 139 of handle 102 may contribute to reducing gaps between these components. For example, as lock 106 is secured onto the handle 102, the complementary tapered surfaces may create a close fit, potentially minimizing spaces therebetween. This configuration may allow for a more precise alignment of components, which in some cases could enhance the overall stability of the assembly of medical device 100.

Lock 106 may include one or more slits 140 extending longitudinally along at least a portion of lock 106. These slit(s) 140 may allow for some flexibility or expansion of lock 106 during assembly or disassembly of medical device 100. For example, slit(s) 140 may allow for lock 106 to expand or flex such that engagement feature 146 of lock 106 can engage with complementary engagement feature 148 of handle 102 in a snap-fit manner. As lock 106 is advanced over distal portion 122, the slit(s) 140 may permit lock 106 to temporarily expand until locking feature 146 passes over and engages with complementary locking feature 148, at which point lock 106 may contract back to its original position, creating a secure snap-fit connection between lock 106 and handle 102. In other aspects, slit(s) 140 may be omitted. In such cases, lock 106 may comprise a sufficiently flexible or semi-flexible material to accommodate the snap-fit assembly and disassembly while still providing a secure connection.

Lock 106 may also include a straight central surface 144 between proximal internal surface 138 and distal internal surface 142. Straight central surface 144 may provide a region for shaft 104 to pass through lock 106. The geometry of these surfaces (e.g., proximal internal surface 138, distal internal surface 142, and central surface 144) may contribute to the overall coupling mechanism that secures shaft 104 to handle 102 and may be complementary to a geometry of distal portion 122 of handle 102.

In some aspects, lock 106 may include one or more surface features 150, e.g., gripping features to assist a user in manipulating lock 106 during assembly or disassembly of medical device 100. Surface features 150 may help the user to effectively secure or release the connection between shaft 104 and handle 102.

Aspects of this disclosure include methods assembling medical device 100. For example, medical device 100 may be assembled by connecting handle 102 to shaft 104 via the coupling mechanism that includes lock 106. In some aspects, the assembly process may begin by aligning the proximal end 126 of shaft 104 with distal portion 122 of handle 102. For example, the flared portion 128 of shaft 104 may abut the distal end 124 of handle 102, allowing the internal surface 130 of shaft 104 to engage with the distal end 124 of handle 102. During this alignment, lumen(s) 105 of shaft 104 may be positioned to align with lumen(s) 112 of handle 102, establishing fluid communication between these lumens. In some cases, alignment features such as cutout(s) 132 on the flared portion 128 of shaft 104 may engage with complementary protrusions on distal end 124 of handle 102, or vice versa, to assist in proper positioning and prevent unwanted rotation. Additionally or alternatively, cutout(s) 132 may enable proximal end 126 of shaft 104 to expand to receive distal end 124 of handle 102, thereby creating a close fit therebetween.

Lock 106 may then be positioned over shaft 104 and moved proximally towards handle 102. Central opening 136 of lock 106 may accommodate both shaft 104 and at least a portion of the distal portion 122 of handle 102. As lock 106 is advanced, proximal internal surface 138 may engage with proximal end 139 of handle 102 and internal distal surface 142 may engage with proximal end 126 of shaft 104. In some aspects, lock 106 may be designed to flex or expand slightly during assembly via slit(s) 140. This flexibility may allow engagement feature 146 on lock 106 to engage with a complementary engagement feature on the distal portion 122 of handle 102. Upon engagement, lock 106 may secure shaft 104 to handle 102, e.g., creating a fluid-tight connection between proximal end 126 of shaft 104 and distal end 124 of handle 102.

Once fully assembled, the components of medical device 100 may be securely connected, allowing for use while maintaining the ability to disassemble for cleaning, replacement, or other purposes.

The configuration described herein with respect to medical device 100 provides for a modular approach, allowing for shaft 104 to be coupled and de-coupled from handle 102. For example, the ability to separate shaft 104 from handle 102 may allow for increased flexibility in component selection and utilization. For example, a medical professional may have access to a range of shafts 104 having a variety of lengths, diameters, and/or durometers, which can be interchanged with a single handle 102. This modular approach may enable the use of different shaft configurations for various procedures or patient anatomies, while maintaining familiarity with a consistent handle design. Accordingly, in some aspects, handle 102 may be designed for multiple uses, undergoing sterilization between procedures, and shaft 104 may be intended for single-use applications.

Furthermore, this modular approach may offer advantages in terms of manufacturing. The ability to produce handles and shafts separately may allow for more efficient production processes and potentially reduce overall manufacturing costs. For example, the interaction between components of medical device 100 as described herein may also contribute to manufacturing efficiencies. In some aspects, the design may allow for increased tolerances in component manufacturing, potentially simplifying production processes or reducing material waste. This may lead to cost savings or improvements in production throughput.

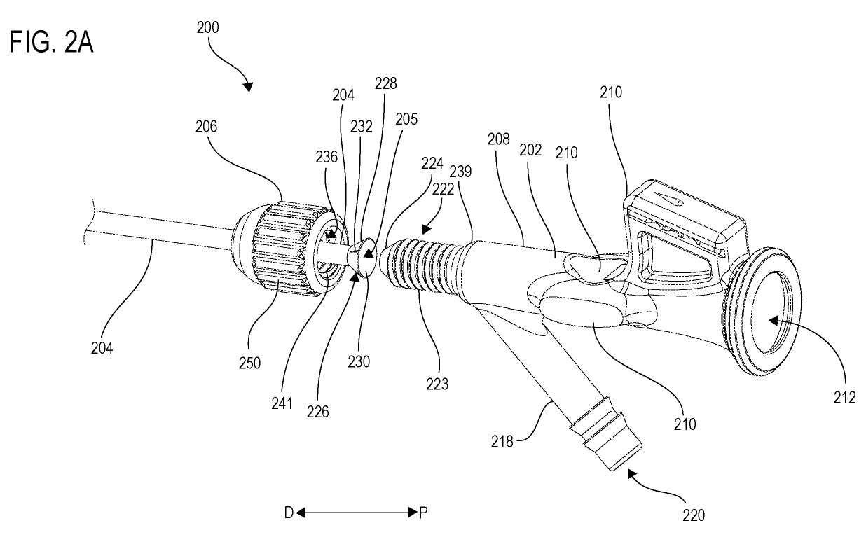

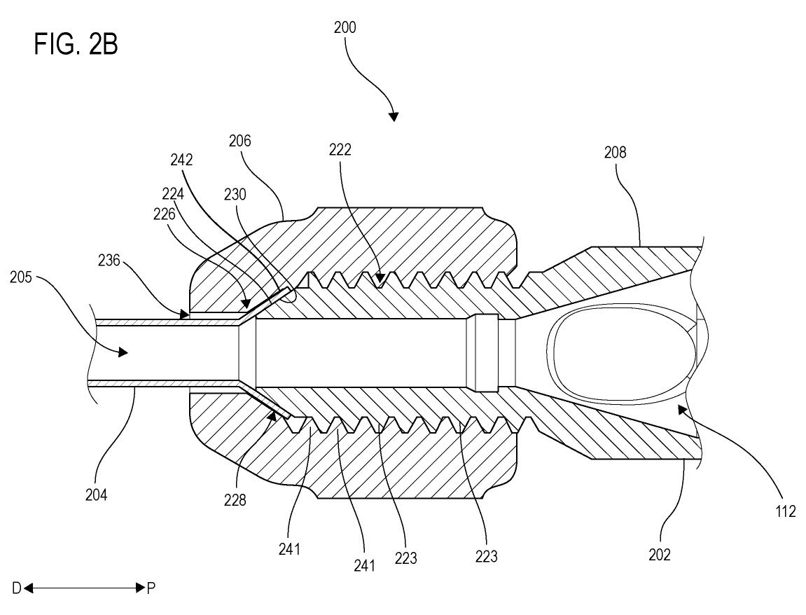

FIGS. 2A and 2B illustrate another exemplary medical device 200 that includes a coupling mechanism to attach a handle to a shaft. FIG. 2A illustrates an exploded view of medical device 200, and FIG. 2B illustrates an assembled cross-sectional view of a portion of medical device 200. Medical device 200 may have any or all of the characteristics of medical device 100, except as described below. For example, medical device 200 may include a handle 202, a shaft 204, and a coupling mechanism that includes a lock 206. In this example, lock 206 incorporates a screw-fit mechanism as opposed to the snap-fit mechanism described above with respect to lock 106 of medical device 100.

Referring primarily to FIG. 2A, handle 202 may include a gripping portion 208 having one or more surface features 210 to promote grip by a hand of a user. Handle 202 may include one or more lumens 212 extending from a proximal end of handle 202 to a distal end of handle 202. In particular, handle lumen(s) 212 may extend through at least a portion of handle 202. Handle 202 may further include a port 218 having an opening 220 in fluid communication with lumen(s) 212.

A distal portion 222 of handle 202 may include a plurality of external threads 241. External threads 223 may be configured to engage with complementary receiving threads 241 of lock 206. This threaded interface may allow for a secure, screw-fit connection between lock 206 and handle 202.

Proximal end 226 of shaft 204 and/or distal end 224 of distal portion 222 may have any or all of the characteristics of proximal end 126 of shaft 104 and/or distal end 124 of distal portion 122 described above with respect to FIGS. 1A and 1B. For example, a proximal end 226 of shaft 204 may include a flared portion 228 having an internal surface 230. Internal surface 230 of shaft 204 may be configured to abut a distal end 224 of distal portion 222 of handle 202. Proximal end 226 may further include cutout(s) 232 configured to assist with coupling to distal end 224. In other aspects, distal end 224 may include protrusions configured to be received within cutout(s) 232 of proximal end 226. Such a configuration may allow for alignment of shaft 204 relative to handle 202.

Similar to lock 106, lock 206 may include an opening 236 that extends from a proximal end of lock 206 to a distal end of lock 206. Opening 236 may accommodate shaft 204 and at least a portion of distal portion 222 of handle 202 as shown in FIG. 2B. Opening 236 of lock 206 may include a plurality of receiving threads 241. As described above, receiving threads 241 may be configured to interact with the plurality of external threads 223 of distal portion 222. This threaded interface between lock 206 and handle 202 may allow for a secure connection when the components are assembled. Lock 206 may further include one or more surface features 250, which may assist a user in gripping and manipulating lock 206 during assembly or disassembly of medical device 200.

Upon assembly, flared portion 228 may be disposed between distal end 224 of handle 202 and an internal distal surface 242 of lock 206. This arrangement may help secure shaft 204 in place and create a fluid-tight seal between the components. The positioning of flared portion 228 between these two surfaces may contribute to the overall stability of the connection and may help maintain proper alignment of the shaft 204 with handle 202. Furthermore, upon assembly, lumen(s) 212 of handle 202 may be in fluid communication with one or more lumen(s) 205 of shaft 204.

This screw-fit design of medical device 200 may provide a secure and adjustable connection between components, allowing for fine-tuning of the assembly tightness and quick assembly. The threaded interface may also facilitate easier disassembly for cleaning or component replacement.

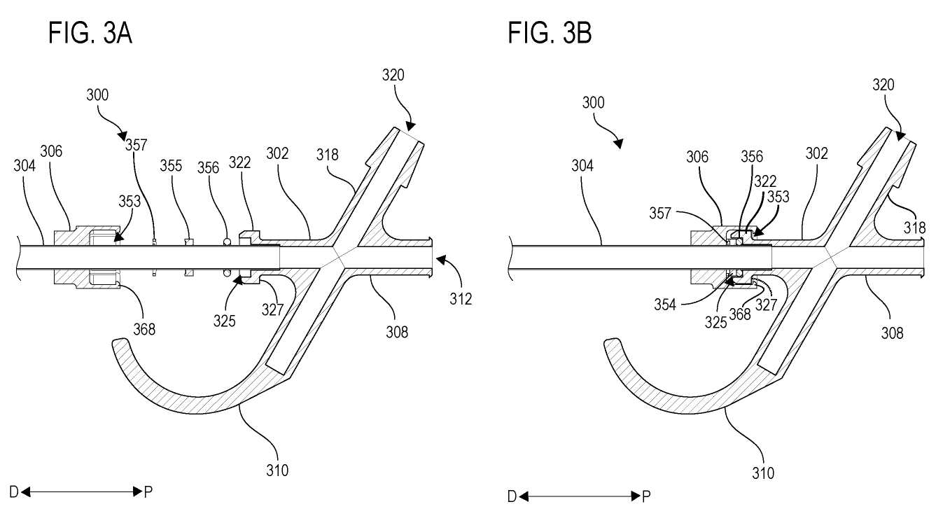

FIGS. 3A and 3B illustrate cross-sectional views of another exemplary medical device 300 with a coupling mechanism to attach a handle to a shaft. Medical device 300 may have any or all of the characteristics of medical device 100 and/or 200, except as described below. For example, medical device 300 may include a handle 302, a shaft 304, and a coupling mechanism that includes a lock 306. At least a proximal portion of shaft 304 may be received within a distal portion of handle 302. Handle 302 may include a gripping portion 308 having one or more surface features 310 to facilitate gripping by a hand of a user. A lumen 312 extends longitudinally through handle 302. Handle 302 may further include a port 318 with an opening 320 that is in fluid communication with lumen 312.

Handle 302 includes a distal portion 322 having a distal-facing recess 325. In some aspects, distal portion 322 may have a cross-sectional dimension (e.g., diameter) greater than a cross-sectional dimension (e.g., diameter) of handle 302, including gripping portion 308. Lock 306 may include a proximal-facing recess 353 configured to receive distal portion 322 of handle 302. A proximal end of lock 306 may include a lip 368 to assist with securing lock 306 to distal portion 322. For example, lock 306 may receive distal portion 322 within proximal-facing recess 353 such that lip 368 engages with a proximal face 327 of distal portion 322. In some aspects, lip 368 may be configured to flex or deform during assembly to allow distal portion 322 to pass through before snapping into place. This configuration may provide an additional securing aspect of coupling mechanism to help maintain the connection between lock 306 and handle 302.

In some implementations, lip 368 may be continuous around the circumference of lock 306, or it may be segmented. The shape and dimensions of lip 368 may be designed to complement the geometry of proximal face 327, to promote a more secure fit. Additionally, the interaction between lip 368 and proximal face 327 may provide tactile or audible feedback to the user during assembly, indicating when the components are properly connected.

Medical device 300, e.g., coupling mechanism of medical device 300, may further include an O-ring 356, a spacer 355, and a washer 357. In some aspects, O-ring 356 may be proximal to both spacer 355 and washer 357. Spacer 355 may be between washer 357 and O-ring 356. Accordingly, when medical device 300 is assembled, O-ring 356, spacer 355, and washer 357 may be between a distal face of proximal-facing recess 353 and a proximal face of distal-facing recess 325. In this configuration, spacer 355 may compress O-ring 356, which in turn may secure shaft 304 in place. For example, compressing O-ring 356 may result in a radially inward force applied to shaft 304 and a radially outward force applied to the walls of distal-facing recess 325. Accordingly, shaft 304 may be secured in place. Washer 357 may assist in positioning O-ring 356 and spacer 355, and may help maintain the relative positions of these components during assembly of medical device 300, e.g., for effective sealing and attachment of shaft 304 to handle 302.

The configuration of medical device 300 may also allow for flexibility in removing or replacing shaft 304. In some aspects, the arrangement of components of coupling mechanism, e.g., lock 306, O-ring 356, spacer 355, and washer 357, may facilitate the attachment/detachment of shaft 304 from handle 302. This mechanism may enable medical professionals to more easily swap out shafts of different lengths, diameters, or materials to suit various procedures and/or patient anatomies. Additionally, the ability to remove shaft 304 may simplify cleaning and sterilization processes of handle 302, potentially extending the usable life of handle 302. In cases where shaft 304 may be damaged or worn, this configuration may allow for quick replacement without replacing the entire medical device 300. This may lead to cost savings and reduced downtime in clinical settings.

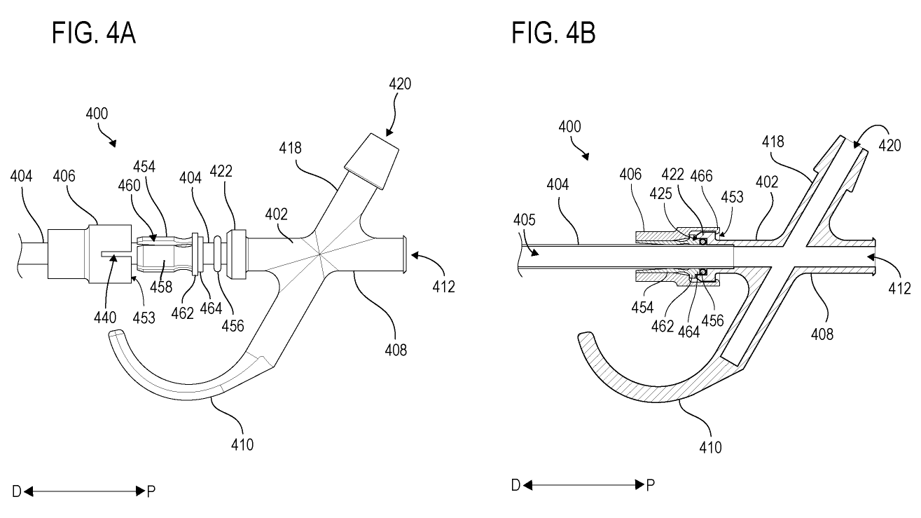

FIGS. 4A and 4B illustrate different views of another exemplary medical device 400. Medical device 400 may incorporate any or all of the characteristics of medical devices 100, 200, and/or 300, except as described below. For example, medical device 400 may include a handle 402, a shaft 404, and a coupling mechanism that includes a lock 406. In some aspects, shaft 404 may extend at least partially into a proximal end of handle 402.

Handle 402 may include a distal portion 422 with a proximal-facing recess 425 configured to receive components of the coupling mechanism. Handle 402 may include a gripping portion 408 and a port 418. A lumen 412 may extend longitudinally through handle 402, and a lumen 420 of port 418 may be in fluid communication with lumen 412 of handle 402. Handle 402 may further include one or more surface features 410 to assist a user with grasping handle 402.

The coupling mechanism of medical device 400 may further include components that work together with lock 406 to secure shaft 404 to handle 402. For example, the coupling mechanism of medical device 400 may include an O-ring 456 and a crimp ring 454. O-ring 456 may be proximal of crimp ring 454. O-ring 456 and crimp ring 454 may assist in securing shaft 404 in place. For example, upon assembly, O-ring 456 may be received within proximal-facing recess 425 of handle 402. A proximal extension 464 of crimp ring 454 may be at least partially received within proximal-facing recess 425, for example to assist with pressing O-ring 456 proximally into proximal-facing recess 425. In aspects, proximal extension 464 may assist with at least partially deforming O-ring 456 within proximal-facing recess 425 such that O-ring 456 presses radially outward against the walls forming proximal-facing recess 425 and radially inward against shaft 404. Such an interaction may create a tight fit so as to secure shaft 404 to handle 402. Such an interaction may create a secure fit between shaft 404 and handle 402, for example, without the use of adhesives or epoxies.

A radial protrusion 462 of crimp ring 454 may be distal of proximal extension 464. Radial protrusion 462 may be configured to engage with a distal end of distal portion 422 of handle 402, for example, to prevent crimp ring 454 from being over-inserted into distal portion 422 of handle 402.

Crimp ring 454 may include a plurality of arms 458 that may be flexible and configured to engage with shaft 404 or lock 406 of the coupling mechanism to provide a secure connection. For example, arms 458 may be received within an opening of lock 406 and flex radially inward, towards shaft 404. The interaction of arms 458 with shaft 404 may assist in securing shaft 404 to lock 406. In some aspects, arms 458 may include one or more cutouts, e.g., slits 460, that allow for increased flexibility during assembly and disassembly.

Lock 406of the coupling mechanism may include a lip 468 to assist with securing handle 402 to shaft 404 via lock 406. For example, lock 406 may receive distal portion 422 such that lip 468 engages with a proximal face 423 of distal portion 422. In some aspects, lip 468 may be configured to flex or deform slightly during assembly to allow distal portion 422 to pass through before snapping into place. This configuration may provide an additional aspect of coupling mechanism to help maintain the connection between lock 406 and handle 402.

Lock 406 may include one or more slits 440 extending longitudinally along at least a portion of lock 406. These slits 440 may allow for some flexibility or expansion of lock 406 during assembly or disassembly of medical device 400. For example, as lock 406 is advanced over distal portion 422, the slits 440 may permit lock 406 to temporarily expand until lip 468 passes over distal portion 422 and engages with proximal face 423, at which point lock 406 may contract back to its original position, creating a secure snap-fit connection between lock 406 and handle 402. In other aspects, slits 440 may be omitted, and lock 406 may comprise a sufficiently flexible or semi-flexible material to accommodate the snap-fit assembly and disassembly while still providing a secure connection.

Medical device 400 may offer a combination of secure attachment and potential modularity of shaft 404 and handle 402. The multiple components of the coupling mechanism, e.g., lock 406 including arms 458 and various lip features, may provide a robust connection while potentially allowing for easier assembly and disassembly. This configuration may enable medical professionals to interchange shafts as needed while maintaining a secure and reliable connection during use.

A number of implementations have been described. Nevertheless, it will be understood that various modifications may be made without departing from the spirit and scope of the disclosure. Accordingly, other implementations are within the scope of the following claims.

What is claimed is:

1. A medical device, comprising:
a handle having a distal portion;
a shaft having a proximal end configured to engage with the distal portion of the handle; and
a coupling mechanism including a lock configured to receive the distal portion of the handle to secure the shaft and the lock to the handle.

2. The medical device of claim 1, wherein the distal portion of the handle comprises a tapered distal end, and wherein the proximal end of the shaft includes a flared portion, wherein the flared portion is configured to abut the tapered distal end of the handle.

3. The medical device of claim 2, wherein the flared portion includes one or more cutouts extending longitudinally along at least a portion of the flared portion.

4. The medical device of claim 2, wherein the flared portion includes one or more cutouts configured to receive a protrusion of the distal portion of the handle.

5. The medical device of claim 1, wherein the lock of the coupling mechanism includes a central opening configured to receive both the shaft and the handle.

6. The medical device of claim 1, wherein the handle is attached to the shaft by the coupling mechanism, and the proximal end of the shaft is disposed between an internal surface of the lock and the distal portion of the handle.

7. The medical device of claim 1, wherein the lock includes one or more indentations configured to engage with a protrusion of the distal portion of the handle to secure the lock to the handle.

8. The medical device of claim 1, wherein the lock comprises one or more slits extending longitudinally along at least a portion of the lock.

9. The medical device of claim 1, wherein the distal portion of the handle includes threads complementary to threads of the lock.

10. The medical device of claim 1, wherein the lock includes a proximal-facing recess configured to receive the distal portion of the handle.

11. The medical device of claim 1, wherein the distal portion of the handle includes a distal-facing recess.

12. The medical device of claim 1, wherein the coupling mechanism further comprises an O-ring.

13. The medical device of claim 1, wherein the coupling mechanism further comprises an O-ring, a spacer and a washer, wherein the spacer is between the washer and the O-ring.

14. The medical device of claim 1, wherein the coupling mechanism further comprises an O-ring and a crimp ring, wherein the crimp ring is distal of the O-ring.

15. The medical device of claim 1, wherein the handle includes a first lumen extending longitudinally therethrough, wherein the shaft includes a second lumen extending longitudinally therethrough, and wherein, upon securing the shaft to the handle, the first lumen is in fluid communication with the second lumen.

16. A medical device, comprising:
a handle having a distal portion with a tapered distal end;
a shaft having a proximal end with a flared portion that receives the tapered distal end of the handle; and
a coupling mechanism including a lock that secures the shaft to the handle, wherein the flared portion of the shaft is between an internal surface of the lock and the tapered distal end of the handle.

17. The medical device of claim 16, wherein the flared portion includes one or more cutouts extending longitudinally along at least a portion of the flared portion.

18. The medical device of claim 16, wherein the shaft is detachable from the handle by manipulating the coupling mechanism to detach the lock from the handle or the shaft.

19. A medical device, comprising:
a handle having a distal portion with a tapered distal end;
a shaft having a proximal end with a flared portion that receives the tapered distal end of the handle; and
a coupling mechanism including a lock, an O-ring, a spacer, and a washer, wherein the coupling mechanism secures the shaft to the handle.

20. The medical device of claim 19, wherein the spacer is between the washer and the O-ring.
